SEQUENCE LISTING (2 sequences)

SEQ ID NO: 1 - DNA, 25 bp, Artificial
  Probe sequence to be hybridized with a target sequence
cgtacgatcgatgtagctagcatgc

SEQ ID NO: 2 - DNA, 25 bp, Artificial
  Target sequence to be hybridized with a probe sequence
gcatgctagctacatcgatcgtacg

(19) United States
(12) Patent Application Publication
(10) Pub. No.: US 2008/0131970 A1
(43) Pub. Date: Jun. 5, 2008

(54) BIOCHEMICAL REACTION CARTRIDGE

(75) Inventor: Wataru Kaku, Yokohama-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/943,317

(22) Filed: Nov. 20, 2007

(30) Foreign Application Priority Data
Dec. 5, 2006 (JP) 2006-328004

(51) Int. Cl. G01N 33/00; B01J 19/00

(52) U.S. Cl. 436/2; 422/68.1

(57) ABSTRACT

A cartridge for conducting uniform biochemical reaction without complicated operations while preventing evaporation of a sample solution is provided. An evaporation preventing substance that is solid at ordinary temperature and melted in the temperature range for biochemical reaction is held inside the cartridge. During biochemical reaction, the melted evaporation preventing substance is present at an interface between the sample solution and a gas phase to prevent the evaporation of the sample solution.

### BACKGROUND OF THE INVENTION

1. Field of the Invention

The present invention relates to a method for determining whether a target substance is present or not in a sample solution obtained from a specimen by using biochemical reactions such as antigen-antibody reaction and nucleic acid hybridization reactions. More particularly, the present invention relates to a cartridge for performing such biochemical reactions.

2. Description of the Related Art

Vigorous efforts have been made to develop analytical and diagnostic methods using gene information as nucleic acid amplification methods as typified by PCR or base-sequencing machines are widespread. A DNA microarray is a representative example. A DNA microarray is an array wherein a plurality of DNA probes with different base sequences is immobilized on a solid-phase carrier such as a slide glass with a high density in an alignment. A DNA microarray is a useful approach for determination of a base sequence of a nucleic acid on a slide glass, detection of variation or polymorphism, analysis of gene expression profile, and the like. A DNA microarray is prepared by applying photolithography technique, such as a method for synthesizing a DNA probe on a substrate with one base-by-one base, and a method for spotting previously-synthesized DNA probes on a substrate by an inkjet method or a pin method (see, for example, U.S. Pat. Nos. 5,424,186 and 5,807,522). A DNA microarray prepared by these methods has several hundreds to hundreds of thousands of kinds of DNA probes immobilized highly densely, thus enabling one analysis to provide extremely large volume of information.

In order to utilize the above features of DNA microarray, it is important that highly-densely immobilized DNA probes are brought into uniform contact with a sample solution. For uniform contact with a sample solution, it is necessary that the sample solution is free from bubbles and has a constant thickness of a liquid layer. To solve these problems, use of a surface-treated cover glass is disclosed (see, for example, Japanese Patent Application Laid-Open Nos. 2002-262854 and 2004-286702). In these methods, a sample solution is dropped on an array surface having DNA probes immobilized, and then the array is covered with a cover glass, a surface of which is treated to have hydrophilic property, thereby enabling the sample solution to have a uniform contact with the array surface.

Further, in hybridization reaction, a small amount of a sample solution is kept a relatively high temperature for a long period, so it is an important problem to prevent the evaporation of the solution during the reaction. To solve this problem, a method wherein an opening of a cartridge is physically closed is disclosed (see Japanese Patent Application Laid-Open No. 2005-300460). In this method, a sample solution is injected into a cartridge through an opening, and paraffin wax having a temperature greater than or equal to its melting point is dropped into the opening. Thereafter, the paraffin wax is cooled to a temperature less than the melting point and solidified, thereby preventing the evaporation of the solution.

Use of a cover glass or closing of an opening of a cartridge as mentioned above can prevent the evaporation, thereby enabling hybridization reaction to be performed with a small amount of a sample solution. However, in the method wherein a cover glass is used, it is difficult to fix the cover glass at a predetermined position. In the worst case, the cover glass is broken and a valuable sample may be wasted. Further, in the method wherein an opening of the cartridge is closed with a solid substance, when a substance for closing the opening is dropped after the injection of a sample solution, complicated working or a special apparatus is required. Furthermore, since the opening is closed with the solid substance, the sample solution filled in the cartridge cannot be stirred and thus the sample solution cannot be uniformly diffused. This may result in adverse effects such as lower reproducibility or a longer period of reaction.

### SUMMARY OF THE INVENTION

An object of the present invention is to provide a cartridge which enables a sample solution to be brought into uniform contact with nucleic acid probes highly densely immobilized on a carrier while holding the sample solution externally operable and preventing evaporation of the solution, and a method of using the cartridge.

In order to solve the above problems, the present invention provides the following cartridge for biochemical reaction.

According to a first embodiment of the present invention, there is provided a biochemical reaction cartridge having a reaction chamber for conducting a biochemical reaction for detecting a target substance, wherein the cartridge holds a substance for preventing evaporation of a sample solution containing the target substance on a wall surface forming the reaction chamber or a space communicating with the reaction chamber.

According to a second embodiment of the present invention, there is provided a method for using the biochemical reaction cartridge of the first embodiment, including heating the cartridge to a temperature equal to or higher than the melting point of the substance for preventing the evaporation, and then conducting a biochemical reaction.

According to the present invention, even when nucleic acid probes are highly densely immobilized, a simple operation allows uniform contact between the nucleic acid probes and a sample solution. This enables uniform hybridization reaction among DNA probes, resulting in the obtainment of highly reliable data.

Further features of the present invention will become apparent from the following description of exemplary embodiments (with reference to the attached drawings).

### BRIEF DESCRIPTION OF THE DRAWINGS

FIG. 1 shows members constituting a biochemical reaction cartridge according to the present invention;

FIG. 2A is a top external view showing a biochemical reaction cartridge according to one embodiment of the present invention;

FIG. 2B is a bottom external view showing the biochemical reaction cartridge according to the embodiment of the present invention; and

FIG. 3 is a cross sectional view of the biochemical reaction cartridge according to the present invention.

### DESCRIPTION OF THE EMBODIMENTS

Hereafter, specific embodiments of a biochemical reaction cartridge of the present invention and a hybridization method using the cartridge will be described.

According to an exemplary embodiment of the present invention, a biochemical reaction cartridge comprises a reaction chamber having a holding space capable of holding a sample solution inside the cartridge; at least two openings; a detection site having detection probes immobilized; and a flow channel for communicating between the openings and the reaction chamber.

The holding space is a space inside the cartridge, which holds a sample solution and provides a reaction field for biochemical reaction.

The opening is a hole for communicating between the outside and the reaction chamber, which can be used for injection and disposal (if necessary) of the sample solution. When a sample solution is injected, an opening for the injection and an opening for discharge air during the solution injection are necessary. Thus, there are provided at least two openings. As long as the opening has a diameter that permits the injection and disposal of the sample solution by use of a liquid control unit such as a pipette tip or a syringe pump, the opening may has any diameter. From a viewpoint of preventing contamination, the opening preferably has a smaller diameter.

The detection site having detection probes immobilized is a site, on which probes capable of specific interaction with a target substance in the sample solution are immobilized in an alignment. As such a detection site, a probe-immobilized carrier can be used. The detection site can be disposed at a desired position exposed to the holding space as long as the detection probes can be brought into contact with the sample solution. The detection site can be disposed at the desired position so as to form preferably at least a part of a face of the cartridge, more preferably at least a part of a bottom face of the cartridge. For example, a probe-immobilized carrier as the detection site can be used as a bottom face of the cartridge.

A material for a carrier having the probes immobilized thereon is not particularly limited as long as the material does not inhibit the interaction between the probes and the target substance. A glass or plastic flat plate is often used. Further, biochemical reactions between target substances in the sample solution and probes, typified by hybridization reaction, are often conducted at a high ion concentration and a high temperature for a long period, and thus strong immobilization of a probe on a carrier is desired. Particularly, the immobilization is preferably attained by covalent binding. Specifically, the immobilization of a probe on a carrier is preferably attained via a linker so as to avoid steric hinderance caused by immobilization.

The flow channel communicating the opening with the holding space is a space required for filling the injected sample solution from the opening to the holding space or sucking the reacted solution from the opening for disposal. Since a sample solution remains in the flow channel as a dead volume, the flow channel preferably has as small a volume as possible.

When a biochemical reaction cartridge having the above construction is used, the sample solution injected from the opening passes through the flow channel communicating the opening with the holding space and is filled in the holding space with a certain volume. This allows the sample solution to be brought into uniform contact with the array surface having probes immobilized thereon. In order to conduct biochemical reaction while keeping the sample solution in uniform contact with the array surface, it is necessary to prevent the evaporation of the sample solution during the reaction.

As a material for the cartridge of the present invention, a high-polymer material having a good moldability is desirably used when the cartridge is produced with molded parts. Examples thereof include plastics such as polycarbonate resin, polyolefin resin, poly acrylic resin, polypropylene resin and polyethylene resin.

According to the present invention, a substance for preventing the evaporation works as a block on a gas-liquid interface between the sample solution and an external gas phase, thereby preventing the evaporation of the sample solution. Specifically, usable is a substance that forms a liquid film between the sample solution and an external air. More specifically, used is a hydrophobic substance that has a melting point in the temperature range from 40° C. to 100° C., at which biochemical reactions are performed, and has a smaller specific gravity than the sample solution. Further, the melting point of the hydrophobic substance is preferably equal to or lower than the lowest temperature among biochemical reaction temperatures.

Since the hydrophobic substance has a smaller specific gravity than the sample solution and is hydrophobic, the substance is not mixed with the hydrophilic sample solution such as a nucleic acid solution at a temperature equal to or higher than the melting point. Thus, the substance remains at an interface between the sample solution and a gas phase to form a film. Then, the substance can function as a blocking cover at the gas-liquid interface. Unlike methods for blocking an opening by a seal or a solid substance, this substance can work as a direct blockage at the gas-liquid interface between the sample solution and the gas phase, thus enabling effective prevention of the evaporation. Further, the hydrophobic substance as a blockage at the gas-liquid interface is in a liquid state at a temperature equal to or higher than the melting point, and thus the sample solution can be stirred during biochemical reaction. This allows uniform diffusion of substances in the solution, resulting in highly reproducible and efficient biochemical reaction.

The above hydrophobic substance can cover a gas-liquid interface between a sample solution and a gas phase during biochemical reaction. However, the present invention is featured by fixing in advance the hydrophobic substance inside a biochemical reaction cartridge for simplifying operations. Examples of the hydrophobic substance to be fixed on an inner wall surface of the cartridge include waxes and greases. Among these, waxes that are less adhered to a solid surface than greases are preferable, considering easy penetration by a pipette, etc. when the substance is in a solid state, and biochemical reaction and subsequent detection. A wax is an organic matter that is generally solid or semisolid at ordinary temperature and has an alkyl group. A melted wax is a liquid having a smaller density than water. There are various kinds of waxes such as natural waxes (Japan wax, jojoba oil, beeswax, montan wax, paraffin wax), synthetic waxes (polyethylene wax, montan wax derivative, hydrogenated castor oil), blended waxes and mixture compositions thereof. Paraffin wax is suitably used since it has a melting point lower than the temperature range for biochemical reactions in the present invention. Paraffin wax is a wax substance having a melting point of around 50° C. Paraffin wax is heated to a temperature equal to or higher than the melting point to become liquid, and the liquid paraffin wax is dropped. Thereafter, the paraffin wax is cooled to a temperature equal to or lower than the melting point, so that paraffin wax can be fixed at a desired position. Paraffin wax can be fixed at any position as long as the position permits paraffin wax to be in contact with a sample solution after paraffin wax is melted. Thus, paraffin wax may be fixed on any of an opening, a holding space, or a flow channel communicating between the opening and a detection site, or on all the inner wall surfaces formed inside a cartridge. Particularly, paraffin wax is preferably fixed on or around the opening so that paraffin wax can be easily positioned at an interface between a sample solution and an external gas phase when the paraffin wax is in a liquid state. Paraffin wax fixed on the opening can be easily penetrated by a point of a pipette tip or the like at the time of injecting a sample solution.

In addition, an evaporation preventing substance is preferably used so that a reaction chamber before its use is hermetically sealed.

As described above, a biochemical reaction cartridge having paraffin wax held on its inner wall surface is heated to a temperature equal to or higher than the melting point of paraffin wax before or after the injection of a sample solution, and thereby the paraffin wax can be re-melted. When paraffin wax is made into a liquid state before biochemical reaction, the paraffin was can function as a blocking cover at a gas-liquid interface between the sample solution and a gas phase.

When paraffin wax is disposed on the inner wall surface or inside the reaction chamber, a temperature increase after sample injection enables an evaporation preventing substance to be simply and immediately disposed at a sample interface.

From a viewpoint of handleability, a sample solution is prepared as an aqueous solution using a specimen. When a specimen is an aqueous solution, the specimen can be used with no treatment or diluted with water or various buffer solutions to prepare a sample solution. When a specimen is a cell or a living tissue, a component that is expected to contain a target biological polymer is extracted from the specimen and dissolved in water or various buffer solutions to prepare a sample solution.

In a method for using a biochemical reaction cartridge of the present invention, the cartridge may be heated by direct contact with a heat source from a viewpoint of heat efficiency. Examples of the heat source include a heater and a peltiert device. Further, the temperature of the cartridge may be controlled by using a thermostatic bath or an incubator.

### Embodiments

Hereafter, the present invention will be described in detail by referring to Embodiments, but the present invention is not limited thereto.

(Preparation of Biochemical Reaction Cartridge)

(1) Preparation of Probe-Immobilized Array

Aminosilane coupling agent (tradename: KBM-903; available from Shin-Etsu Chemical Co., Ltd.) was dissolved so as to have a concentration of 0.1 wt %, and stirred for 30 minutes for hydrolysis of methoxy group. The aqueous solution was applied onto an alkali-washed synthetic quartz glass substrate by a spin coater, and then baked in an oven at 120° C. for one hour. Next, 2.7 mg of N-maleimidocaproyloxy succinimide (available from Dojindo Laboratories; hereafter abbreviated as EMCS) was weighed and dissolved in a solution of dimethylsulfoxide (DMSO) and ethanol (1:1), preparing an EMCS solution having a final concentration of 0.3 mg/mL. The EMCS solution was applied onto the baked amino group-introduced substrate by a spin coater so that a maleimide group was introduced onto a surface of the substrate.

A synthetic single strand DNA probe (sequence A, see Table 1) having a mercapto (SH) group introduced therein was dissolved in an aqueous solution containing 7.5 wt % of glycerin, 7.5 wt % of urea and 1.0 wt % of acetylene alcohol (tradename: Acetylenol E100, available from Kawaken Fine Chemicals Co., Ltd.) so as to have a concentration of 0.6 OD.

TABLE 1

DNA probe sequence

| | Sequence A |
|---|---|
| DNA probe | 5′ HS(CH₂)₆OP(O₂)O— SEQ ID NO: 1 3′ |

In the sequence A, HS represents a mercapto group.

The probe-containing solution was spotted on the maleimide group-introduced substrate by an inkjet method. After the spotting, the substrate was allowed to stand still in a chamber with constant temperature and humidity for conducting reaction between maleimide group on the substrate surface and mercapto group at the terminal of the DNA probe. After 30 minutes, the substrate was washed with 1M-NaCl/50 mM phosphate buffer solution (pH 7.0), washed lightly with pure water, and then dried by blowing nitrogen gas. The substrate was attached to an adhesive sheet, and cut by a diamond cutter into pieces having an 18-mm square, providing an 18-mm square probe-immobilized array 11.

(2) Cartridge Assembling

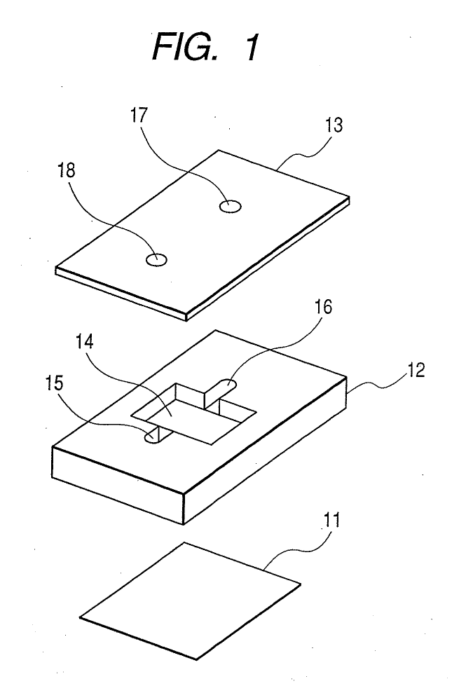

FIG. 1 shows a cartridge main member 12 and a cartridge cover member 13, which were prepared by injection molding with a metal mold using a polycarbonate resin. The cartridge cover member 13 has openings 17 and 18 used for injection and disposal of a sample solution, and air discharge. Paraffin wax that had been heated to 70° C. to be in a liquid state was dropped on the openings 17 and 18 of the cartridge cover member 13 and their surrounding areas. AmpliWax PCR Gem 50 (Applied Biosystems) was used as the paraffin wax. Thereafter, the paraffin wax was cooled to 25° C. and solidified to close the openings 17 and 18. The paraffin wax closing the openings can be penetrated with an object having a sharp tip such as a pipette tip, thus enabling easy injection of a sample solution. The cartridge main body 12 has a space 14 for holding a sample solution and flow channels 15 and 16 formed by bonding the probe-immobilized array 11 and the cartridge cover member 13 thereto. The cartridge cover member 13 is bonded to the cartridge main body 12 by a method such as ultrasonic welding method, laser welding method, or an adhesive so that air tightness is secured. The probe-immobilized array 11 is bonded to the cartridge main body 12 by using an adhesive such as an ultraviolet curable resin or a visible light curable resin so that air tightness is secured. According to the above processes, a biochemical reaction cartridge 21 having an evaporation preventing substance fixed thereon was obtained. In this embodiment, fixation of paraffin wax was conducted before a cartridge is assembled, but the fixation may be conducted after the assembling of a cartridge.

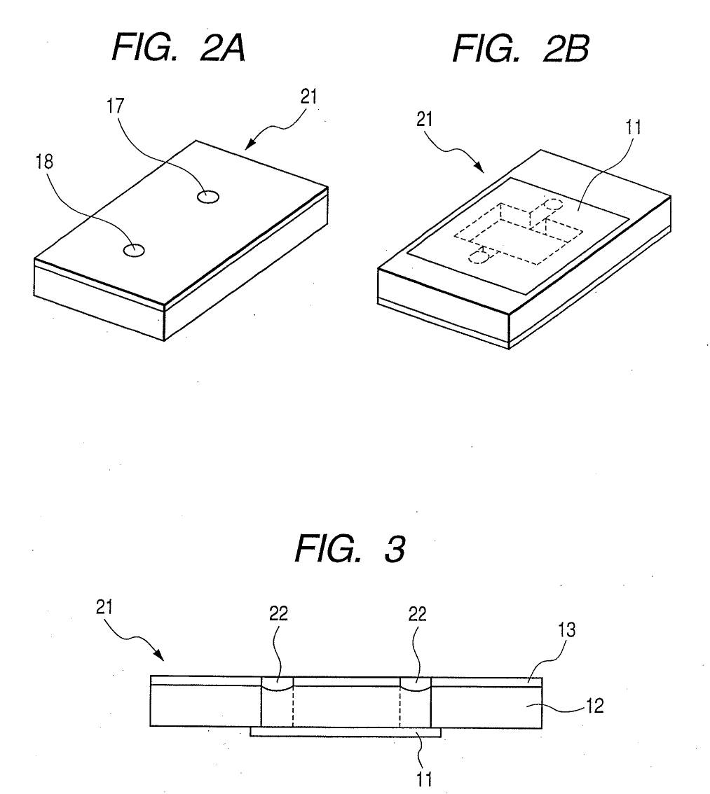

FIGS. 2A and 2B show a biochemical reaction cartridge 21 prepared by assembling the cartridge main body 12, cartridge cover member 13, and probe-immobilized array 11. FIG. 2A is a top external view of the cartridge and FIG. 2B is a bottom external view of the cartridge. Further, FIG. 3 is a cross sectional view of the biochemical reaction cartridge 21. In FIG. 3, numeral 22 denotes an evaporation preventing substance. It should be noted that the number of units each having a reaction chamber, a flow channel, and an opening is not limited to the number indicated in the figure and two or more units may be provided in one cartridge. Thus, the position of a flow channel relative to a reaction chamber can be also modified.

(Hybridization Reaction)

The paraffin wax fixed on the openings 17 and 18 of the biochemical reaction cartridge 21 prepared by the above-mentioned processes was penetrated with a pipette tip so that 100 μL of a sample solution (0.25 nM target nucleic acid, 6× SSPE, 10% formamide and 0.05% SDS) was injected. After the injection of the sample solution, the biochemical reaction cartridge was weighed. The target nucleic acid contained in the sample solution is a nucleic acid having a base sequence described in Table 2, and this nucleic acid is complementary to the nucleic acid immobilized on the probe-immobilized array 11. Further, in order to detect hybridization with the probe immobilized on the array, a fluorescent dye Cy3 is conjugated to the 5′ terminal of the target nucleic acid (Sequence B, see Table 2).

TABLE 2

Target nucleic acid sequence

| | Sequence B |
|---|---|
| Target nucleic acid | 5′ Cy3- SEQ ID NO: 2 3′ |

In the Sequence B, Cy3 represents a fluorescent dye Cy3.

After being weighed, the biochemical reaction cartridge was heated at 92° C. for 5 minutes, and hybridization reaction was performed at 50° C. for 4 hours. When being heated at 92° C., it was confirmed that the paraffin wax fixed on the openings was melted on an upper layer of the sample solution. The cartridge was heated by using DIGITAL HOT PLATE HP-2S (AS ONE). After the hybridization reaction, the weight of the biochemical reaction cartridge was measured again. Thereafter, the sample solution was removed with a syringe pump, and the cartridge was washed twice with 100 μL of a cleaning solution (6× SSPE, 10% formamide and 0.05% SDS). After the cleaning solution was removed by a syringe pump, the biochemical reaction cartridge was dried by blowing air. The dried biochemical reaction cartridge was scanned from its bottom surface by a confocal fluorescence scanner (excitation wavelength: 532 nm, PMT 400V).

(Results)

Results show almost no difference in weight of the biochemical reaction cartridge having the evaporation preventing substance (paraffin wax) fixed thereon between before and after the reaction (see Table 3). Further, results of fluorescence scanning show that uniform signals were obtained from the array (see Table 4).

TABLE 3
Results of weight measurement (unit: g)

| | With paraffin wax | Without paraffin wax |
|---|---|---|
| After injection of | 5.8347 | 5.8845 |
| sample solution | | |
| After termination of | 5.8327 | 5.8595 |
| biochemical reaction | | |
| Weight difference | 0.0020 | 0.0250 |

TABLE 4
Results of fluorescent brightness measurement

| Dot Nos. | Fluorescent brightness value |
|---|---|
| Dot 1 | 14500 |
| Dot 2 | 14580 |
| Dot 3 | 14858 |
| Dot 4 | 14544 |
| Dot 5 | 14454 |
| Dot 6 | 14497 |
| Dot 7 | 14490 |
| Dot 8 | 14438 |
| Dot 9 | 14608 |
| Dot 10 | 14379 |
| Dot 11 | 14467 |
| Dot 12 | 14478 |
| Dot 13 | 14121 |
| Dot 14 | 14296 |
| Dot 15 | 14078 |
| Dot 16 | 14150 |
| Average | 14434 |

These results show that use of a biochemical reaction cartridge of the present invention having an evaporation preventing substance fixed thereon can prevent evaporation of a sample solution during biochemical reaction, thereby enabling the obtainment of stable signals.

The present invention is not limited to the above embodiments and various changes and modifications can be made within the spirit and scope of the present invention. Therefore to apprise the public of the scope of the present invention, the following claims are made.

This application claims the benefit of Japanese Patent Application No. 2006-328004, filed Dec. 5, 2006, which is hereby incorporated by reference herein in its entirety.

What is claimed is:

1. A biochemical reaction cartridge comprising a reaction chamber for conducting a biochemical reaction for detecting a target substance, wherein the cartridge holds a substance for preventing evaporation of a sample solution containing the target substance on a wall surface forming the reaction chamber or a space communicating with the reaction chamber.

2. The biochemical reaction cartridge according to claim 1, wherein the substance for preventing the evaporation is a substance that forms a liquid film between the sample solution and an external air.

3. The biochemical reaction cartridge according to claim 1, wherein the target substance is a nucleic acid.

4. The biochemical reaction cartridge according to claim 1, comprising:
at least two openings communicating with the reaction chamber via a flow channel; and
a detection site provided inside the reaction chamber,
wherein a detection probe for detecting the target substance is immobilized on the detection site and the detection site is disposed so that the detection probe is brought into contact with the sample solution.

5. The biochemical reaction cartridge according to claim 4, wherein the detection probe is a nucleic acid.

6. The biochemical reaction cartridge according to claim 5, wherein the biochemical reaction is a hybridization reaction.

7. The biochemical reaction cartridge according to claim 1, wherein the substance for preventing the evaporation is held on an inner wall surface of any or all of the opening, the reaction chamber and the flow channel.

8. The biochemical reaction cartridge according to claim 1, wherein the substance for preventing the evaporation is a hydrophobic substance that has a melting point in a temperature range from 40° C. to 100° C. for conducting biochemical reaction and a smaller specific gravity than the sample solution.

9. The biochemical reaction cartridge according to claim 8, wherein the hydrophobic substance is paraffin wax.

10. A method of using the biochemical reaction cartridge according to claim 1, comprising:
heating the cartridge to a temperature equal to or higher than the melting point of the substance for preventing the evaporation; and
conducting a biochemical reaction.
